(19) United States
(12) Patent Application Publication
(10) Pub. No.: US 2010/0189747 A1
(43) Pub. Date: Jul. 29, 2010

(54) Compositions and methods for treating or preventing hiv infection

(76) Inventors: Raymond Weinstein, Woodbridge, VA (US); Michael Weinstein, Los Angeles, CA (US); Ken Alibek, Dunn Loring, VA (US)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 10/566,586

(22) PCT Filed: Jan. 28, 2004
(86) PCT No.: PCT/US04/02064

§ 371 Date: Aug. 23, 2007

(30) Foreign Application Priority Data
Jul. 31, 2003 (US) 60491258
Aug. 11, 2003 (US) 60493767
Aug. 22, 2003 (US) 60496908
Sep. 11, 2003 (US) 60501832

(51) Int. Cl. A61K 39/275; A61K 39/12; C12Q 1/70

(52) U.S. Cl. 424/232.1; 424/204.1; 435/5

(57) ABSTRACT

The present invention provides methods and compositions for treating and/or preventing HIV infection in a subject in need thereof. It features the use of poxviruses, such as vaccinia virus, for therapy, prophylaxis, and diagnosis of HIV, as well as for any other medical or veterinary use associated with HIV or homologous viruses. The invention also provides for the use of poxviruses in the discovery of new agents to prevent and/or treat HIV infection.

This application claims the benefit of U.S. Provisional Application Nos. 60/491,258 filed Jul. 31, 2003, 60/493,767 filed Aug. 11, 2003, 60/496,908 filed Aug. 22, 2003, and 60/501,832 filed Sep.11, 2003, which are hereby incorporated by reference in their entirety.

### BACKGROUND OF THE INVENTION

Acquired Immune Deficiency Syndrome (“AIDS”) is one of the most serious health threats confronting the human population today. AIDS is caused by a virus known as human immunodeficiency virus (“HIV”) which presently includes HIV-1 and HIV-2. Over 40 million people are estimated to be living with HIV/AIDS. Current projections suggest that an additional 45 million people will become infected between 2002 and 2010. So far, it is believed that more 25 million people have died from AIDS.

Since its emergence in the 1970s, HIV has produced a continually growing global pandemic, and it has, thus far, defied all attempts to produce an effective vaccine. Although a number of drugs have been developed to treat the disease, all have limited usefulness, serious side effects, a high potential for resistance, and none have been identified so far which can cure or prevent it. HIV vaccine research has expanded over recent years, but success so far using HIV-based components has been limited. See, e.g., Graham et al., J. Inf. Disease., 166:244-252, 1992; Belshe et al., J. Inf Disease., 183:1343-52, 2001; Horton et al., J. Virol., 76:7187-7202, 2002; Gilbert et al., Vaccine, 21:2933-2947, 2003.

### DESCRIPTION OF DRAWINGS

FIG. 1 (A-C). Comparison of cells from vaccinated versus non-vaccinated subjects, infected with the macrophage (CCR5) tropic HIV. A. A comparison of the mean+standard error measurement of the vaccinated versus non-vaccinated groups in cultures without autologous serum. (*, p<0.05) B. A comparison of the mean+standard error measurement of the vaccinated versus non-vaccinated groups in cultures with autologous serum (*, p<0.05; **, p<0.01). C. Comparison of the mean+standard error measurement of cells from vaccinated versus non-vaccinated subjects, infected with the T-cell (CXCR4) tropic HIV.

### DESCRIPTION OF THE INVENTION

The present invention provides methods and compositions for treating and/or preventing HIV infection in a subject in need thereof. It features the use of poxviruses for therapy, prophylaxis, and diagnosis of HIV, as well as for any other medical or veterinary use associated with HIV and homologous viruses. The invention also provides for the use of poxviruses in the discovery of new agents to prevent and/or treat HIV infection.

A poxvirus or a component thereof, can be used to treat and/or prevent infection caused by any virus, preferably a lentivirus, such as HIV, that uses a CCR5 chemokine receptor for its infection of cells. This includes, but is not limited to, e.g., HIV-1 (e.g., clades A, B, C, D, and G, R5 and R5X4 viruses, etc.), HIV-2 (e.g., R5 and R5X4 viruses, etc.), simian immunodeficiency virus (SIV), simian/human immunodeficiency virus (SHIV), feline immunodeficiency virus (FIV), bovine immunodeficiency virus (BIV) (Wright et al., Vet. Res. Commun., 26:239-50, 2002), HTLV-1, HTLV-2, etc. It can be used as a vaccine, adjuvant, therapeutic agent, in combination with other agents, or in any suitable manner to treat and/or prevent such infections.

Any poxvirus can be used in accordance with the present invention, including, but not limited to, orthopoxvirus, parapoxvirus, avipoxvirus, capripoxvirus, leporipoxvirus, suipoxvirus, etc. Orthopoxvirus, include, e.g., buffalopox, camelpox, cowpox, monkeypox, rabbitpox, raccoon pox, tatera pox, canarypox, fowlpox, vaccinia, variola, and vole pox. Vaccinia virus is the prototype of the genus Orthopoxvirus for the desired effects, but other poxviruses can be used in its place. Thus, although the disclosure below may be written in terms of vaccinia, any poxvirus can be utilized in accordance with the present invention.

Vaccinia is a double-stranded DNA (deoxyribonucleic acid) virus. All strains, derivatives, variants, mutations, naturally-occurring strains, genetically-engineered, recombinant, etc., of vaccinia can be used in accordance with the present invention. For more information on vaccinia and other poxvirus, see e.g., Virology, Fields et al., Volume 2, Chapters 74-75, Raven Press, 1990.

An amount of the poxvirus, such as vaccinia virus, can be administered to a subject in a quantity which is effective to achieve a therapeutic or prophylactic effect. The term “poxvirus,” “vaccinia virus,” etc., indicates that the virus (genome and protein coat) is administered to a subject. It can be administered in any effective form, including, e.g., as a live virus, as a live-attenuated virus, as a replication-deficient virus, as a viral extract not having any live viral particles, etc. Compositions comprising a poxvirus can be produced and utilized in any suitable manner, including, e.g., recombinant, naked DNA technology, etc.

The term “treating” is used conventionally, e.g., the management or care of a subject for the purpose of combating, alleviating, reducing, relieving, improving, eliminating, etc., one or more signs or symptoms associated with HIV infection. Treatment includes delaying the progression of HIV and its associated symptoms, thereby extending the life expectancy of an infected subject, and/or delaying or reducing the onset of symptoms associated with HIV infection. Treating can involve inhibiting, reducing, diminishing, etc., the replication and other events in the life cycle of the HIV virus.

The term “preventing” HIV infection indicates that a subject's susceptibility to HIV infection upon exposure to the virus is reduced or diminished as a result of the administration of the poxvirus. The subject's resistance to HIV infection is increased or improved by the poxvirus treatment since s/he is less likely to become infected by the virus. Any amount of improved resistance is useful, e.g., greater than 5-fold, greater than 7-fold, greater than ten-fold, etc., and any such improvement can be regarded as prevention.

A poxvirus, or component thereof, used in the present invention can be prepared routinely, or obtained from commercial sources. Attenuated strains are preferred. Attenuated strains are less able to cause disease, and are considered less virulent and weakened as compared to strains that are not attenuated.

Any strain of vaccinia virus, or components thereof, can be utilized to achieve a prophylactic and/or therapeutic effect, including, but not limited to, e.g., strains available from the ATCC, ECACC, or other virus collections, replication-competent, replication-deficient, non-replicating, attenuated strains, modified vaccinia Ankara (MVA), vaccinia virus Ankara, NYVAC (ATCC No. VR-2559) replication-deficient vaccinia viruses, VV Copenhagen, VV Western Reserve, VV Wyeth (ATCC No. VR325), Elstree, strains deficient in vCCI (Reading et al., J. Immunol., 170:1435-42, 2003), and/or vGF, strains comprising one or more copies of the 17K myristyloprotein, poxvirus strains, CCR5-dependent poxvirus strains, etc. Dryvax®, a vaccinia (smallpox) vaccine currently licensed in the United States, is a lyophilized, live-virus preparation of infectious vaccinia virus (Wyeth Laboratories, Inc., Marietta, Pa.). Other strains which have been used include, but are not limited to, e.g., Lister, Bordeaux, Paris, Massachusetts 999, New York, Temple of Heaven, Patwadangar, Ikeda, Bern, Vienna, Bohemia, Finland, Hamburg, Budapest, Aosta, Spain, Sweden, B-51, Tashkent, EM-63, LE-IVP (Lister), etc. See, also, Smallpox and its Eradication, Fenner et al., WHO, Geneva, 1988, e.g., Chapter 11. Other strains include, e.g., MVA-BN (modified vaccinia Ankara—Bavarian Nordic) (ECACC V00083008; WO 02/42480), MVA-Vero (US 20030013190), MVA-NH, MVA 572 (ECACC V94012707), LC16m8, and ACAM1000 (ATCC Deposit No. PTA-3321; WO 02/085411). Any strain of canarypox can be utilized as well, including attenuated canarypox virus such as, e.g., ALVAC (ATCC No. VR-2547).

Deposited strains also include, e.g., ATCC Nos. VR-117 (CL), VR-118 (Lederle-Chorioallantoic), VR-119 (WR (Mouse Neurotropic), VR-1354 (WR (NIH TC-adapted), VR-1431 (P-4), VR-1441 (IHD-W), VR-1508 (Modified vaccinia virus Ankara (MVA)), VR-1536 (New York City Department of Health Laboratories (Wyeth-calf adapted)), VR-1549 (Elstree (Lister Vaccine)), VR-156 (IHD), VR-2010 (AS), VR-2031 (Vtk-79), VR-2034 (S-variant), VR-2042 (vP-7), VR-2043 (vP-9), VR-2292 (SLZ103[recombinant Vaccinia virus (WR)]), VR-2379 (Rpmuhr+[recombinant of Utrecht strain Rpuhr23]), VR-2589 (VVtm1:hPC1 [recombinant Vaccinia virus, in vitro construct]), VR-302 (Brighton), VR-3103 (IHD-W Dts 16 [Vaccinia ts-mutant]), VR-3109 (IHD-W Dts 46 [Vaccinia ts-mutant]), VR-3110 (IHD-W Dts 2 [Vaccinia ts-mutant]), VR-3113 (IHD-W Dts 17 [Vaccinia ts-mutant]), VR-3121 (IHD-W Dts8 [Vaccinia ts-mutant]), VR-3126 (IHD-W Dts 33 [Vaccinia ts-mutant]), VR-3129 (IHD-W Dts 48 [Vaccinia ts-mutant]), VR-3130 (IBD-W Dts 4 [Vaccinia ts-mutant]), VR-3139 (IHD-W Dts 50 [Vaccinia ts-mutant]), VR-3142 (IHD-W Dts 10 [Vaccinia ts-mutant]), VR-3144 (IHD-W Dts20), VR-3147 (IHD-W Dts 35 [Vaccinia ts-mutant]), VR-3148 (IHD-W Dts 40), VR-3154 (IHD-W Dts71 [Vaccinia ts-mutant]), VR-3160 (IHD-W Dts52 [Vaccinia ts-mutant]), VR-3161 (IHD-W Dts 57), VR-3165 (IBD-W Dts 77), VR-3166 (IHD-W Dts 82), VR-3169 (IHD-W Dts97 [Vaccinia ts-mutant]), VR-3175 (IHD-W Dts 78 [Vaccinia ts-mutant]), VR-3176 (IHD-W Dts 83 [Vaccinia ts-mutant]), VR-3178 (IHD-W Dts 93 [Vaccinia ts-mutant]), VR-3196 (IHD-W Dts 95 [Vaccinia ts-mutant]), VR-587 (Yaba monkey tumor virus deposited as Yaba monkey tumor virus, Yatapoxvirus (Roswell Park-Yohn)), VR-838 (Raccoonpox virus, Orthopoxvirus (Herman)).

A vaccinia virus is a preferred poxvirus in accordance with the present invention, but other poxviruses can also be used to treat and/or prevent HIV. For example, any poxvirus which expresses a gp120-like or TAT-like polypeptide, or which depends on CCR5 for entry into a cell can be used in accordance with the present invention.

Vaccinia virus can be administered to subjects according to any regimen which is effective for treating and/or preventing HIV infection. The particular dosages (i.e., effective amounts), and number and frequency of vaccinations can be determined routinely.

An effective amount of virus, or virus component, is the quantity of virus, or virus component, which is useful to achieve the desired purpose, e.g., to treat and/or prevent HIV infection. These amounts can be determined routinely. Effective amounts can be the same or less than the amounts currently used to achieve pox immunity with a pox vaccine. For example, Dryvax™ is commonly used at a potency of 100 million pock-forming units (pfu)/ml for primary vaccination for smallpox. Any effective amount can be used in accordance with the present invention, e.g., about 10⁵-10⁹ pfu/ml. The quantities of the particular virus which is utilized can be adjusted and determined routinely, e.g., to eliminate or reduce adverse reactions associated with the virus, as well as depending on the health of the patient receiving the treatment.

The specific dose level and frequency of dosage may vary, and can depend upon a variety of factors, including the activity and state of the specific poxvirus, e.g., whether it is live, heat-inactivated, attenuated, etc., its metabolic stability and length of action, rate of excretion, mode and time of administration, and the age, body weight, general health, gender, diet, and particular condition of the subject undergoing treatment or prevention.

Poxvirus can be administered in any form by any effective route, including, e.g., oral, parenteral, enteral, intraperitoneal, topical, transdermal (e.g., using any standard patch), ophthalmic, nasally, local, non-oral, such as aerosal, spray, inhalation, percutaneous (epidermal), subcutaneous, intravenous, intramuscular, buccal, sublingual, rectal, vaginal, intra-arterial, mucosal, and intrathecal, etc. It can be administered alone, or in combination with any ingredient(s), active or inactive.

Any subject can be administered a poxvirus in accordance with the present invention, including subjects who have been exposed to HIV, but have not yet developed HIV infection, as well as subjects who have progressed to one or more of the clinical symptoms of HIV infection (e.g., AIDS). In addition to treating and/or preventing HIV infection in humans, a poxvirus can be used to treat other organisms (e.g., non-human primates, cats, etc.) infected with HIV, or HIV-related viruses, such as SIV, SHIV, or FIV. Thus, subjects who can be treated include, e.g.; mammals, humans, monkeys, apes, chimpanzees, gorillas, cats, dogs, mice, rats, etc.

Subjects, who have been exposed to HIV virus, or who are at risk for developing the disease, are particular candidates for poxvirus vaccination. For instance, a subject who has not yet tested positive, but has been exposed to HIV, can be administered vaccinia virus as a prophylactic/therapeutic approach. Individuals at high-risk for the disease, such as sexually-active individuals, subjects in parts of the world where HIV infection is high, subjects receiving blood and/or other invasive medical procedures, can also receive vaccination to increase their resistance to HIV infection.

In addition to administering the whole poxvirus, components of it can also be administered in accordance with the present invention. By the phrase “component,” it is meant any part of the virus, which is less than the whole virus genome, including particular nucleic segments of its genome, as well as any product which is produced using the viral genome. This includes modifications to polypeptides encoded for by the virus.

Components include polypeptides comprising the virus, such as envelope proteins, processing enzymes, structural proteins, nucleic acid synthesis enzymes, glycoproteins, carbohydrates, lipids, antigens or antigenic fragments of the virus, etc. Also included are nucleic acid fragments of the whole genome, including fragments comprising complete gene sequences, control sequences, etc.

Components includes one or more of the over about 198 open reading frames (ORF) and about 268 genes that have been identified in vaccinia and other poxvirus. Components include one or more of the genes and products thereof described in, but not limited to, Antoine et al., Virology, 244:365-396, 1998, and Goebel et al., Virology, 179(1):247-266, 1990 for vaccinia virus; Willer et al., Virology, 264(2):319-43, 1999 for Leporipoxvirus Shope fibroma virus (SFV); Cameron et al., Virology, 264(2):298-318, 1999 for myxoma virus; Shchelkunov et al., Virology, 297(2):172-94, 2002 for monkeypox virus; Shchelkunov and Totmenin, Virus Genes, 9(3):231-45, 1995 for variola, Massung et al., Virology, 201(2):215-40, 1994. For example, the polypeptide coding for the 17K myristylprotein, and which has amino acid sequence homology to gp120, can be used alone or in combination with other antigens, etc., in accordance with the present invention. See, e.g., Antoine et al., 1998; Barrett et al., Seminars in Immunol., 13:73-84, 2001. See, also Tables 1 (from Goebel et al., Virol., 179:247-266, 1990) and 2 (from Antoine et al., Virol., 244:365-396, 1998). Moreover, one or more of the aforementioned genes and open reading frames can be deleted from a vaccinia virus, e.g., to eliminate a toxic or other undesirable effect of an administered virus.

A useful composition can comprise one of the components of a poxvirus, including one or more of the components described in Tables 1 and 2. These can be individual purified and then combined into a therapeutic or prophylactic composition, or extracts can be prepared from viral particles and treated as desired. The individual components can be purified from the viral particles, or produced recombinantly, e.g., where a target gene is cloned, expressed in a host cell under conditions where the polypeptide is manufactured by the cell, and separating and purifying the polypeptide accordingly to conventional methods. Components can also be administered as naked DNA. See, e.g., U.S. Pat. No. 6,413,942.

The therapeutic and/or prophylactic effect achieved with the poxvirus can be independent of an immunological response to it. For example, the purpose of ordinary smallpox vaccination is to elicit an immune response by the host. This response is both humoral and cellular, involving the generation of specific antibodies and immune cells (such as T-cells, cytolytic or cytotoxic T lymphocytes, etc.) which protect a host from future invasion by the smallpox virus. While the present invention is not bound by any mechanism through which the poxvirus achieves its therapeutic and/or prophylactic effect, it can be mediated through a pathway separate from the immune response and not require cellular or humoral immunity. For example, poxvirus, or a component thereof, can directly block or inhibit the ability of a HIV to infect a cell. In this respect, the poxvirus, or component of it, acts as an antagonist, blocker, etc., of HIV's ability to infect target cells. HIV usually activates a G-protein-coupled signal pathway cascade. Poxvirus can interfere with this pathway or modify it such a way that the cell is more difficult to infect, thereby increasing its resistance to the HIV virus. Consequently, the effective amounts of a poxvirus, or component thereof, can differ from the amounts that are ordinarily used when the objective is to achieve a humoral and/or cellular immune response.

Vaccination with vaccinia can be associated with adverse reactions. Those at highest risk include, e.g., pregnant women, immunocompromised patients (e.g. HIV-positive), and persons who have atopic dermatitis or eczema. Strains which are attenuated or otherwise modified to reduce adverse effects are especially useful in accordance with the present invention, e.g., for administration to persons at risk for adverse effects.

Modified strains of vaccinia can be utilized that are deficient, mutated, engineered, etc., in one or more of the about 198 open reading frames (ORF) and/or about 268 genes that comprise vaccinia (depending on the strain or variant). In addition, genes can be inserted into vaccinia, including, one or more copies of a vaccinia gene of interest (e.g., 17K myristylprotein, vCCI), and/or genes coding for all or part of an HIV proteins, such as gp120 or gp40.

The present invention also provides methods of treating and/or preventing HIV infection in a subject in need thereof, comprising, e.g., administering multiple doses of a poxvirus, or components thereof, to a subject, wherein each dose is administered at a time interval from the previous dose, and are effective to maintain a therapeutic effect, or to maintain protection against HIV infection. As discussed above, a dose of the poxvirus, or component thereof, is the amount of virus which is useful for accomplishing the therapeutic or prophylactic effect. More than one dose can be administered to the subject in order to maintain the therapeutic efficacy of the treatment, or to maintain protection against HIV infection. For example, smallpox immunization is usually achieved by a single vaccination with a booster every 5-10 years. To maintain protection against HIV, more frequent vaccination can be used, e.g., multiple times a year, at least twice a year, yearly, every two years, every three years, more than once every less than five years, more than once every less than ten years, etc. The periods between the separate and sequential vaccinations can be referred to as “time intervals.” These intervals can be spaced apart by any desired time period which is effective to maintain protection or therapeutic efficacy in treating an infected subject. The intervals can be predetermined or preset, where they are already specified, or they can be determined by monitoring the progress of a subject, e.g., using blood serum to measure poxvirus antibody titer, or HIV titer in an infected subject. The frequency of vaccination utilized to achieve efficacy may vary depending upon multiple factors, including, e.g., person-to-person variations in the immune system, the stage of HIV infection, the potency of the virus or vaccine, etc, and may be as often as every 3 months to once every 5 years.

The present invention also provides methods of treating and/or preventing lentivirus infection in a subject in need thereof, comprising: administering an effective amount of a poxvirus or component thereof, wherein said amount is effective to treat and/or prevent lentiviral infection, with the proviso that a lentivirus nucleic acid, such as HIV, is not contained in the poxvirus genome. This excludes, e.g., a poxvirus which is utilized as a vector to administer HIV nucleic acid, such as when HIV nucleic acid is inserted into the poxvirus genome.

The present invention also provides methods of identifying a component of a poxvirus, or a poxvirus-associated agent, which interferes with HIV infection, and components and agents identified thereby. Interfering with HIV infection indicates that the agent or component decreases, reduces, diminishes, lessens, etc., the ability of a susceptible cell or organism to become infected with HIV virus as compared to the same cell or organism in the same conditions, but in the absence of the agent or component. Interference with HIV infection can occur at any level, e.g., by blocking the ability of HIV to attach to its receptor(s) on a cell, by blocking the ability of HIV to be taken into a cell, by blocking viral function once inside the cell, by blocking viral infection, etc. The invention is not limited by the mechanism through which HIV interference is achieved. By interfering with HIV infection, the cell's or organism's resistance to HIV is increased.

These methods can involve one of more of the following steps in any effective order, e.g., (1) contacting a cell or organism which is susceptible to HIV infection with poxvirus, or a component thereof, or a poxvirus-associated agent, (2) contacting said cell or organism with HIV under conditions effective for said HIV to infect said cell or organism, and, (3) (a) determining whether said cell or organism is resistant to HIV infection, whereby said agent is identified as interfering with HIV infection, or (3) (b) identifying the poxvirus, or component thereof, which confers resistance to HIV infection. The term “organism” as used herein indicates a fully-gestated animal.

The method can also involve a step of identifying the poxvirus, or a component thereof, as the agent which confers resistance to HIV infection. Identifying the poxvirus, or component thereof, which confers resistance to HIV infection, indicates that the poxvirus is positively determined or ascertained to provide protection or resistance against HIV. This indicates a positive result in the method.

Agents can be tested for their ability to interfere with HIV infection in any suitable system, including whole animals and cell culture. Animal cells useful in the present invention are those which are susceptible to HIV infection, i.e., they are capable of being infected by the HIV virus. They can be naturally-susceptible, or genetically-engineered to confer susceptibility, e.g., by expressing HIV receptor (CCR5, CD4, etc.), or by grafting on the human immune system. Any methods for testing whether a cell or organism is infected with HIV can be used, e.g., measuring anti-HIV antibody titer (e.g., gp120 antibodies), reverse transcriptase protein or nucleic acid, or any other polypeptide or nucleic acid.

Any suitable animal model for testing the efficacy and dosage of a poxvirus (or component thereof) can be used in accordance with the present invention. These include, but are not limited to, e.g., SCID mice reconstituted with human immune system components (e.g., peripheral blood lymphocytes) [e.g., Zhang et al., Proc. Natl. Acad. Sci., 93:14720-14725, 1996, using SCIC.bg mice], chimpanzees infected with HIV-1, macaque monkeys infected with SIV, HIV2, or chimeric SIV/HIV [e.g., Johnson, Curr. Opin. Immunol., 8(4):554-560, 1996], cats infected with feline immunodeficiency virus, HIV-1 transgenic mouse model [e.g., mice which have integrated molecular clone pNL4-3 containing 7.4 kb of the HIV-1 proviral genome deleted in the gag and pol genes (Dickie et al., Virology, 185:109-119, 1991; transgenic mice carrying an HIV provirus, optionally with deletion of one or more HIV genes (Tinkle et al., J. Clin. Invest., 100(1):32-9, 1997)], HIV-1 transgenic rat model, human CD4 transgenic rat model, horse infected with EIAV, sheep infected with visna virus, goats infected with CAEV, etc. See, also, The Retroviridae, J. A. Levy, ed., Plenum Press, 1993, e.g., Chapters 3, 4, and 5.

A vaccinia virus-associated agent is any substance which is produced in response to a vaccinia infection, or in response to inhalation, injection, ingestion, etc., of any vaccinia virus, or component thereof. This substance can be present in a culture medium in which cells exposed to vaccinia have been cultured, or can be present in blood serum when harvested from an organism exposed to vaccinia. The present invention provides compositions which comprise such substances.

The invention also provides combinations of pharmaceutical agents for treating and/or preventing HIV, e.g., poxvirus, or a component thereof, and an agent which is used to treat HIV, such as a protease inhibitor or a reverse transcriptase inhibitor. Examples of the latter classes of drug, include, but are not limited to, saquinavir, ritonavir, indinavir, nelfinavir, amprenavir, lopinavir, atazanavir, fosamprenavir, tipranavir, AZT, ddI, ddC, ddT, 3TC, nevirapine, delavirdine, etc. The active agents can be present in the same dosage unit (e.g., a composition), or can be used as separate dosage units.

In addition, a poxvirus, such as vaccinia, can be administered in combination with HIV nucleic acid. The HIV nucleic acid can be physically joined to the poxvirus genome, or it can be administered as a separate component. For example, HIV nucleic acid (e.g., coding for gp 120 or another viral antigen) can be administered at the same time as a poxvirus, but as a physically separated entity, or it can be administered at subsequent times after receiving only poxvirus) as part of a regimen for treating and/or preventing HIV infection.

The present invention also provides methods of making a poxvirus composition for conferring resistance to HIV infection or treating HIV infection, comprising, one or more of the following steps in any effective order, e.g., preparing a composition comprising poxvirus, or a poxvirus component thereof, and/or identifying that the poxvirus, or component thereof, confers resistance to, or treats, HIV infection. As mentioned earlier, the identifying step indicates obtaining a positive result in finding that the poxvirus (e.g., vaccinia), or component thereof, provides resistance, protection, treatment, etc., against the HIV virus.

The preparation of a poxvirus composition can be carried out routinely, e.g., according to conventional methods used for vaccine manufacture. Preparing includes culturing poxvirus, isolating poxvirus, putting poxvirus into a form suitable for administration (oral, injection, nasal, etc.), making poxvirus components recombinantly, etc. The prepared poxvirus (or components of it) can be assayed for its ability to confer resistance to HIV infection to an organism challenged with it or provide a therapeutic effect. By this, it is meant that a sample of the prepared composition is tested to determine its titer, concentration, potency, etc., in making a subject, to whom it is administered, “resistant” to the HIV virus, or for its therapeutic effect. The assay step can be carried out on every batch, or only selected batches, etc. A purpose of this step is, e.g., to confirm that the manufactured poxvirus possesses an anti-HIV activity for which it is to be administered. Any suitable assay or testing method can be utilized, e.g., in vitro methods of evaluating its efficacy or potency. For instance, the determining step can involve, e.g., challenging said organism, or cells derived from it, with infectious HIV, and detecting the expression in said organism or cells of gp120, HIV reverse transcriptase, p24, infectious HIV particles, and/or HIV nucleic acid. By “challenge” it is meant the cells or organism are placed in contact with the HIV virus under conditions which are effective to become infected by it. These conditions will vary, depending upon how the assay is specifically accomplished.

When poxvirus is administered to a host, it can elicit a cellular response that is responsible or associated with the host's subsequent ability to resist HIV infection and/or treat HIV infection. This response can be measured, and used as index or marker to assess the efficacy of the poxvirus, and/or to determine effective amounts of it for the desired purpose (i.e., treating or preventing HIV infection). The appearance of one or more of the following “markers” can be modulated (e.g., elicited, stimulated, down-regulated, up-regulated, etc) by poxvirus, and associated with its anti-HIV effect, thereby making the marker useful as an indicator of poxvirus efficacy. By the term “marker,” it is meant any measurable response to a poxvirus, including its effect on HIV's ability to infect and replicate in a cell, as well as on the host's immune system and the cells which comprise it. These markers, include, but are not limited to, one or more of the following agents, activities, responses, pathways, etc.:

CD4 expression, e.g., measuring the amount of CD4 present in a cell-type that is susceptible to HIV infection

HIV coreceptor expression, e.g., CCR5 or CXCR4 chemokine receptor, including its cell-surface expression

Cytokine receptors

Virus-specific CTLs (cytolytic or cytotoxic T-cells, including CD8+ T-cells) which are capable of lysing HIV infected cells (cells can be co-infected with poxvirus and HIV, or infected by HIV alone)

CD8 cells

Cytokines, including mediators and regulators of innate immunity, such as interferons, type I interferon, interleukins, interleukin-15, interleukin-12, tumor necrosis factor, interleukin-1, interleukin-6, interleukin-10, etc.; and mediators and regulators of specific immunity, such as interleukins, interleukin-2, interleukin-4, transforming growth factor-beta, interferon-gamma, lymphotoxin, interleukin-5, etc.

Chemokines (a large family of structurally homologous cytokines, that, e.g., stimulate leukocyte motility and directed movement), including, but not limited to, the C—C and C—X—C families. Examples of chemokines, include, but are not limited to, e.g., interleukin 8, Gro, platelet basic protein, epithelial-derived neutrophil attractant 78, platelet factor 4, interferon-gamma-induced protein 10, stromal cell-derived factor-1, monocyte chemotactic proteins 1, 2, and/or 3, RANTES, monocyte inflammatory protein 1-alpha and 1-beta (“MIP”), eotaxin, lymphotaxin, etc.

Th1/Th2 phenotype and cytokine secretion pattern. Effector T-cells (e.g., CD4+ helper T-cells) can be categorized, on the basis of the cytokines they secrete, into Th1 and Th2 cells. Th1 cells secrete, e.g., interferon-gamma, lymphotoxin-alpha, TNF-beta, IL-2, IL-10, and CCR5 ligands, such as RANTES and MIPS. Th2 cells secrete, e.g., IL-4, IL-5, IL-6, IL-9, IL-10, IL-13, etc. Th1 and Th2 cells also include resting, but polarized T-cells (i.e., committed to a Th type). In addition to cytokine production profiles, there are a number of cell surface markers that can be used to differentiate between Th1 and Th2 subtypes. For example, Th1 cells express both components of IL-12 receptor chains (beta1 and beta2), while Th2 cells exhibit IL-12R-beta1. Th2 cells exhibit both IFN-gamma receptor chains (a and b), while Th1 cells express IFN-gamma-R-alpha. Th2 cells appear to express a fully functional IL-1 receptor, and ST2L/T1, an IL-1R-like molecule, is found on Th2 cells. Chemokine receptors CXCR-3 and CCR-5 are also characteristic of Th1 cells, while CXCR-4, CCR-3, CCR-4, CCR-7 and CCR-8 are associated with Th2 cells. CD30, a member of the TNF superfamily, is associated with Th2 cells. The Th1/Th2 pattern can be polarized by poxvirus administration, resulting in a phenotype that favors the secretion, etc., of cytokines that inhibit HIV infection and/or render cells resistant to infection. One or more of the aforementioned molecules can be utilized as markers of poxvirus efficacy

Antibodies that specifically recognize HIV, e.g., neutralizing antibodies

Antibodies that specifically recognize poxvirus

Complement control protein. Vaccinia virus encodes a secreted complement control protein (VCP, 35-kDa) protein with sequence homology to the SCR-containing complement control protein superfamily. It binds C3b and C4b, and interferes with the complement cascade by providing cofactor activity for the cleavage of C3 and C4 by factor I, and by accelerating the decay of the C3 converse of both the alternative and, more effectively, the classical pathway of complement activation. VCP may suppress the complement system or their receptor expression, rendering the host less susceptible to the complement-enhancement of HIV infection

Activation state of a cytokine receptor, e.g., CCR5 receptor or other HIV chemokine coreceptor. For example, poxvirus can interfere with CCR5 activation after HIV binding, e.g., by modulating tyrosine kinase feedback pathways

One or more of the vaccinia proteins listed in Tables 1 and 2. This includes any poxvirus-encoded protein that specifically interferes with CCR5/CD4/gp120 interactions, including, e.g., vaccinia encoded CC chemokine binding proteins and/or IFN-gamma receptor-like protein

RNA interference with HIV expression/replication in infected cell

Alpha-defensins 1, 2, and/or 3

Soluble factors including those produced by CD8+ lymphocytes and sometimes referred to as CAF

Interference with the HIV life cycle, including viral entry, import into the host cell nucleus, viral integration into host genome, Rev-dependent and Rev-independent transport from the host nucleus, replication, gene expression, RNA splicing, etc

Inhibiting HIV replication, including its ability to make copies of itself in the cell, and for productive viral particles to be extruded into the blood

Inhibiting the ability of HIV to infect a cell, e.g., to bind to CD4 and/or its coreceptor, for the envelope protein to fuse with the host cell membrane, etc.

Modulating gene expression of the HIV virus, including modulating regulatory genes (e.g., tat and rev), accessory genes (e.g., vif, vpu, vpr, and nef), structural genes (e.g., gag, pol, and env), inner core polypeptides (e.g., gag, p17, p24, p7, and p9), viral enzymes (pol, reverse transcriptase, protease, and integrase), and envelope proteins (e.g., env, gp120, and gp41). The phrase “gene expression” is used broadly to mean any step in the pathway from viral RNA to protein synthesis, and therefore includes all regulatory processes, transcription, translation, polypeptide processing, etc.

Modulating activity of a HIV encoded polypeptide, including, tat, rev, vif, vpu, vpr, nef, gag, p17, p24, p7, p9, pol, reverse transcriptase, protease, integrase, env, gp120, gp41, etc.

Modulating viral regulatory sequences, such as RRE, cis-acting repressive sequences (CRS), and inhibitory/instability RNA sequences (INS)

Any cell or tissue of the immune system, including, but not limited to, lymphocytes, B lymphocytes, T lymphocytes, helper T cells, cytotoxic (or cytolytic) T cells (“CTL), natural killer (NK) cells, naive T cells, memory T cells, CD4+ helper T cells, CD8+ CTLs, monocytes, macrophages, antigen-presenting cells (APCs), dendritic cells, granulocytes, etc.

The present invention also provides kits comprising a poxvirus. For example, a kit for preventing HIV infection, comprising: an effective amount of a poxvirus, and instructions for administering an effective amount of said poxvirus to a subject to prevent HIV infection; and a kit for treating HIV infection, comprising: an effective amount of a poxvirus, and instructions for administering an effective amount of said poxvirus to a subject to treat HIV infection. The instructions can provide any information that is useful for directing the administration of the poxvirus for the desired purpose.

The present invention also provides methods of advertising, licensing, selling, purchasing, etc., a poxvirus for the purpose of treating and/or preventing HIV infection. Methods can comprise, one or more of the following steps in any effective order: e.g., displaying information (a) comprising instructions for administering a poxvirus for treating and/or preventing HIV infection or (b) comprising a description of the use of poxvirus for treating and/or preventing HIV infection, in a printed or computer-readable medium (e.g., on the Web, Internet, personal computer, server, etc); offering for sale a poxvirus for treating and/or preventing HIV infection in a printed or computer-readable medium; accepting an offer to purchase poxvirus for said use in a printed or computer-readable medium. cl Examples

The following experiments were performed in the laboratory of Dr. Beda Brichacek and Dr. Michael Bukrinsky of the Department of Microbiology and Tropical Medicine, The George Washington University, Washington D.C. 20037.

### Methods

### Subject Selection and Specimen Collection.

Twenty subjects were chosen for inclusion in the study. Ten subjects had been immunized with vaccinia within the previous 3 to 6 months, and ten subjects had never been immunized with vaccinia. All subjects were healthy and had a negative HIV test within the previous year. No subjects of northern European descent were used in order to avoid the potentially complicating factor of including a subject who might be homozygous for the CCR5-delta32 mutation. Two tubes of heparinized blood and 1 serum separator tube were collected. All blood samples from all subjects were drawn within 6 hours of each other, and were immediately processed to separate the PBMCs using standard methods of Ficoll-Hypaque centrifugation.

### Cell Culture Preparation.

PBMCs were centrifuged at 1200 rpm for 11 minutes and resuspended in RPMI tissue culture medium+10% fetal calf serum+10 μg/ml gentamicin at a concentration of about 1-3×10⁶ cells/ml with a final concentration of 2×10⁶ cells/culture. Cell cultures were incubated in a CO₂ incubator. On the second day, one of the utilized strains of HIV was mixed with either culture medium or serum from each individual subject and incubated on ice for 7 hours after which 175 μl of each mixture was added to the autologous cell cultures. The next day 1 ml of cell culture media was added and the cultures were incubated for 5 hours to dilute the viral inoculum and to allow the virus to detach. The supernatant was carefully aspirated and 1 ml of fresh media was added before the cultures were spun down at 1000 rpm for 7 minutes. The supernatant was again aspirated and 2 ml of fresh media was added to each culture. 150 μl of supernatant for RT analysis was aspirated from each culture tube on days 2, 5, 8 and 10, and if needed, up to an additional 1 ml was aspirated and replaced with fresh media. On day 2, PHA was added to the tubes of culture series F to act as a cell activator. On day 5, 2 ml of supernatant was removed from each of tubes of culture series F and replaced with 2 ml media+human serum+IL-2.

### Reverse Transcriptase (RT) Analysis.

The measurements of viral replication were performed by standard RT assays using tritiated thymidine as described in numerous articles in the scientific literature. See, e.g., Rey et al., Virology, 181(1), 165-71, 1991.

### Results

All results are based on RT analysis using tritiated thymidine, and are given in counts per minute (CPM).

Culture Series A, the control, demonstrated no viral replication in any cultures.

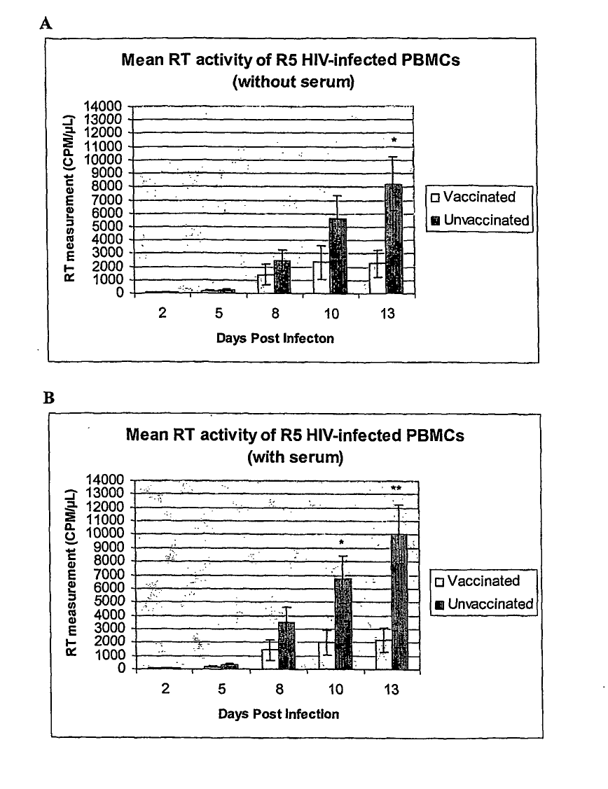

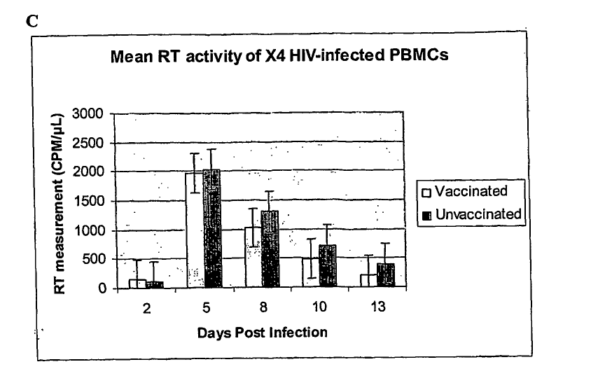

Culture Series B (without serum; FIG. 1A) demonstrated a significant reduction of viral replication in most cultures from vaccinated subjects when compared to unvaccinated subjects. Two subjects (1 and 10) showed a complete lack of viral replication, comparable to the controls in culture series A. One subject was excluded from all analyses when it was subsequently discovered that the subject had had a highly anomalous reaction to the vaccinia immunization with recurrent skin lesions for months afterward. This suggested an inadequate immune response to the vaccinia, and this subject correspondingly did not show any protection against HIV in cell culture, demonstrating viral replication comparable to unvaccinated subjects.

Culture Series C (with serum; FIG. 1B) also demonstrated a significant reduction of viral replication in most cultures from vaccinated subjects, when compared to unvaccinated subjects. The same two subjects (1 and 10) noted in culture series B also had no demonstrable viral replication, comparable to the controls in culture series A. The addition of autologous serum in culture series C further enhanced the difference between vaccinated and unvaccinated subjects when compared to culture series B (no serum).

Culture Series D, E and F, using the T-cell (CXCR4) tropic HIV (FIG. 1C), demonstrated no difference between vaccinated and unvaccinated subjects, including the two subjects (1 and 10) who were resistant to infection by the macrophage (CCR5) tropic HIV in culture series B and C. As stated in the methods section, care was taken in the selection of subjects to avoid those of northern European descent who might be homozygous for the CCR5-delta32 mutation, so this cannot be an explanation for the described resistance. There was also no difference noted between the addition of serum and no serum (cultures D and E).

### Discussion

By at least day 10, there is a statistically significant difference between the vaccinated and non-vaccinated subjects in culture series B and C (p=0.035 and 0.013 respectively) that increases by day 13 (p=0.017 and 0.008 respectively), indicating a resistance to infection by HIV in the vaccinated subjects (FIG. 1). Subjects 1 and 10 demonstrated total resistance to macrophage (CCR5) tropic HIV infection in both culture series B and C, with RT measurements equal to the non-HIV infected control (culture series A). The fact that the same result was achieved in both sets of cultures, while infection was easily achieved with the T-cell (CXCR4) tropic HIV in cultures D, E and F, indicate these finding were not the result of laboratory error.

Without further elaboration, it is believed that one skilled in the art can, using the preceding description, utilize the present invention to its fullest extent. The following preferred specific embodiments are, therefore, to be construed as merely illustrative, and not limitative of the remainder of the disclosure in any way whatsoever. The entire disclosure of all applications, patents and publications, cited above and in the figures are hereby incorporated by reference in their entirety, including of U.S. Provisional Application Nos. 60/491,258 filed Jul. 31, 2003, 60/493,767 filed Aug. 11, 2003, 60/496,908 filed Aug. 22, 2003, and 60/501,832 filed Sep. 11, 2003.

1. A method of preventing HIV infection in a subject in need thereof, comprising:
administering an effective amount of a vaccinia virus, wherein said amount is effective to prevent HIV infection, with the proviso that HIV nucleic acid is not contained within the vaccinia virus genome.

2-5. (canceled)

6. A method of claim 1, wherein said subject has been exposed to HIV virus or is at risk for exposure to HIV.

7. A method of claim 1, further comprising administering a second effective amount of a vaccinia at a predetermined time interval following the administering of the first amount.

8. A method of claim 1, wherein said vaccinia virus is an attenuated vaccinia virus.

9. A method of claim 1, wherein said poxvirus is administered through the mucosa.

10. A method of claim 1, wherein said vaccinia virus utilizes a CCR5 chemokine receptor for entry into a cell.

11. A method of claim 1, further comprising monitoring the HIV status of said subject.

12. A method of claim 1, where said poxvirus has been assayed for its ability to interfere with HIV infection.

13. A method of claim 1, wherein the preventing HIV infection is not a result of an immunological response to a poxvirus antigen.

14-23. (canceled)

24. A method of treating HIV infection in a subject in need thereof, comprising:
administering multiple doses, each having an effective amount of an attenuated vaccinia virus to a subject infected with HIV, wherein said amount is effective to treat HIV infection and wherein each dose is administered at a predetermined time interval from the previous dose, and are effective to maintain protection against HIV infection

25-41. (canceled)

42. A method of making a vaccinia virus, composition for conferring resistance to HIV infection, comprising:
preparing a composition comprising vaccinia virus, or a vaccinia virus component thereof, and
determining that said composition confers resistance to HIV infection to an organism or cell challenged with it.

43. A method of claim 42, wherein said determining whether said composition confers resistance to HIV infection is accomplished by:
challenging said organism, or cell, with infectious HIV, and
detecting the expression in said organism or cells of gp120, HIV reverse transcriptase, p24, infectious HIV particles, and/or HIV nucleic acid.

44-48. (canceled)
